(19) United States
(12) Patent Application Publication
(10) Pub. No.: US 2015/0257659 A1
(43) Pub. Date: Sep. 17, 2015

(54) DEVICE AND METHOD FOR OBTAINING VITAL SIGN INFORMATION OF A LIVING BEING

(71) Applicant: KONINKLIJKE PHILIPS N.V., Eindhoven (NL)

(72) Inventors: Harry Broers, 'S-Hertogenbosch (NL); Joost Adolf Mans, Helmond (NL); Vincent Jeanne, Eindhoven (NL)

(21) Appl. No.: 14/436,204

(22) PCT Filed: Oct. 15, 2013
(86) PCT No.: PCT/IB2013/059353

§ 371 Date: Apr. 16, 2015

Related U.S. Application Data
(60) Provisional application No. 61/717,223, filed on Oct. 23, 2012.

(51) Int. Cl. A61B 5/026; A61B 5/0295; A61B 5/00

(52) U.S. Cl. CPC A61B 5/0261; A61B 5/7278; A61B 5/0295; A61B 5/748; A61B 2576/00; A61B 2562/0233; A61B 2560/0247

(57) ABSTRACT

The present invention relates to a device (1) and method for obtaining vital sign information of a living being (2). The proposed device (1) comprises a detection unit (3) for receiving light (4) in at least one wavelength interval reflected from at least a region of interest (20) of a living being (2) and for generating an input signal (5) from the received light (4), a processing unit (6) for processing the input signal (5) and deriving vital sign information (7) of said living being from said input signal (5) by use of remote photoplethysmography, and an illumination unit (8) for illuminating at least said region of interest (20) during illumination intervals with light, wherein said light during said illumination intervals is optimized for deriving vital sign information from an input signal generated by use of remote photoplethysmography from received light reflected from said region of interest.

### FIELD OF THE INVENTION

The present invention relates to a device and a corresponding method for obtaining vital sign information of a living being.

### BACKGROUND OF THE INVENTION

Unobtrusive vital sign monitoring using a video camera, or remote PPG (photoplethysmography), has been demonstrated and found relevant for patient monitoring. Remote photoplethysmographic imaging is, for instance, described in Wim Verkruysse, Lars O. Svaasand, and J. Stuart Nelson, “Remote plethysmographic imaging using ambient light”, Optics Express, Vol. 16, No. 26, December 2008. It is based on the principle that temporal variations in blood volume in the skin lead to variations in light absorptions by the skin. Such variations can be registered by a video camera that takes images of a skin area, e.g. the face, while processing calculates the pixel average over a manually selected region (typically part of the cheek in this system). By looking at periodic variations of this average signal, the heart beat rate and respiratory rate can be extracted. There are meanwhile a number of further publications and patent applications that describe details of devices and methods for obtaining vital signs of a patient by use of remote PPG.

Thus, the pulsation of arterial blood causes changes in light absorption. Those changes observed with a photodetector (or an array of photodetectors) form a PPG (photo-plethysmography) signal (also called, among other, a pleth wave). Pulsation of the blood is caused by the beating heart, i.e. peaks in the PPG signal correspond to the individual beats of the heart. Therefore, a PPG signal is a heartbeat signal in itself. The normalized amplitude of this signal is different for different wavelengths, and for some wavelengths it is also a function of blood oxygenation.

Although regular video data have been shown to yield adequate vital signs (sometimes also called biometrical signals, such as heartbeat, respiration rate, SpO2 rate, etc.) in many cases, the image acquisition for challenging cases, like strong motion, low light levels, non-white illumination, needs further improvement. The known methods and devices are generally robust to motion and different lighting environment as long as one dominant light source is present. In such condition the PPG technology has proven to be accurate and robust up to a point that it can be used on a treadmill during fitness exercises.

One major problem encountered in image-based (e.g. camera-based) vital signs occurs when no dominant light is present in the environment. Indeed, for indoor application several light sources are generally positioned in the environment e.g. for atmosphere creation. These light sources exhibits similar illumination strength, violating the dominant light source requirement, but different spectrum and hence present different color characteristics. The settings of the light sources could be varying over time which leads to dramatic color change on the measurement area (i.e. the region of interest) of the subject (in particular a living being's face) and make the vital signs extraction extremely challenging and even impossible in some cases.

### SUMMARY OF THE INVENTION

It is an object of the present invention to provide a device and a corresponding method for obtaining vital sign information of a living being having a higher accuracy and reliability, in particular in situations with changing light conditions, compared to known devices and methods.

In a first aspect of the present invention a device for obtaining vital sign information of a living being is presented comprising:
a detection unit for receiving light in at least one wavelength interval reflected from at least a region of interest of a living being and for generating an input signal from the received light,
a processing unit for processing the input signal and deriving vital sign information of said living being from said input signal by use of remote photoplethysmography, and
an illumination unit for illuminating at least said region of interest during illumination intervals with light, wherein said light during said illumination intervals is optimized for deriving vital sign information from an input signal generated by use of remote photoplethysmography from received light reflected from said region of interest.

In a further aspect of the present invention a corresponding method for obtaining vital sign information of a living being is presented.

Preferred embodiments of the invention are defined in the dependent claims. It shall be understood that the claimed method has similar and/or identical preferred embodiments as the claimed device and as defined in the dependent claims.

One possible approach to solve the above described problem is to use constant light conditions aimed at the subject (i.e. the living being, such as a patient or a person, but generally also an animal) being monitored. However, since the application environment is unknown it can generally not be judged if the light conditions are static (e.g. some fitness clubs or households have fancy changing lighting atmosphere), and it is generally also not possible to “prescribe” such constant light conditions.

Hence, according to the present invention a method and device for unobtrusive vital signs monitoring (e.g. heartbeat monitoring, SpO2 monitoring, etc.) using a detections unit, e.g. including a video camera, in conditions with changing color spectrum or intensity of one or more controllable light source(s), generally referred to as illumination unit. The illumination unit is controlled in such way that for a short period (the illumination period) the light, in particular the light spectrum and/or intensity, is optimal for the vital signs monitoring measurement. Thus, only during this illumination period the light reflected from the region of interest (ROI) is measured and/or processed to obtain vital signs. Thus, in an embodiment the light reflected from the ROI is continuously measured, but only light measured during the illumination periods is then processed. In another embodiment, light reflected from the ROI is only measured during the illumination periods.

Preferably, the illumination is such that the light during the illumination periods is invisible for the human eye of a human observer. In the remaining time period the illumination unit is preferably used for atmosphere creation or other purposes.

The present invention thus provides a solution for image-based (camera-based) vital signs extraction in environments with changing light conditions which is enhancing the user experience, reliability and accuracy.

In an embodiment of the present invention the proposed device further comprises a control unit for controlling said detection unit to receive light and/or generate input signals only during said illumination intervals. In another (additional or alternative) embodiment the proposed device further comprises a control unit for controlling said processing unit to process only portions of input signals generated from light received during said illumination intervals. The control units may be separate units or a combined unit. These embodiments lead less required storage space and/or processing time and efforts.

In another preferred embodiment of the present invention the proposed device further comprises a control unit for controlling said illumination unit to illuminate at least said region of interest only during said illumination intervals with light. Thus, the desired illumination can be achieved by e.g. controlling brightness, color, frequency, etc. of the illumination, depending also on the kind of light source(s) provided as illumination unit. Said light sources may e.g. be LEDs, laser diodes, conventional light bulbs, neon lights, etc. which may be controlled. In the simplest embodiment a light source is used that emits the desired light for optimal vital sign measurement.

In a preferred embodiment the proposed device further comprises a control unit for synchronizing the illumination of said at least one region of interest by said illumination unit with the reception of light and/or generation of input signals by said detection unit and/or with said processing of input signal by said processing unit. Thus, an optimized illumination and input signal generation and processing is achieved, even if the illumination of the ROI is not made periodically.

Preferably, said illumination unit is configured to illuminate at least said region of interest during periodic illumination intervals with light and said detection unit is configured to detect said periodic illumination intervals from received light and to subsequently receive light and/or generate input signals only during said periodic illumination intervals. Thus, no separated control means are required to control the detection unit, but the detection unit is able to recognize when the ROI is illuminated and then controls (i.e. synchronizes) itself to the periodic illumination to save power and storage time.

In an advantageous embodiment said illumination unit is configured to control the wavelength of light emitted during said illumination intervals and/or to control the duration of said illumination intervals such that the emitted light during said illumination intervals is invisible or unobtrusive for the human eye. Preferably, said illumination shall not change or disturb the lighting atmosphere, but shall be unrecognizable for the any people present at the place of illumination or in the surroundings.

For this purpose said illumination unit is preferably configured to emit infrared light during said illumination intervals.

In another embodiment said illumination unit is preferably configured for this purpose to emit high frequency light pulses of light in the visible spectral range during said illumination intervals with a low duty cycle. In the latter embodiment a human observer will perceive the illumination as a constant light source with much lower intensity. Above certain frequencies the flicker (introduced by the frequency of the light pulses) will not be visible. Further, by providing that the intensity of the normal illumination is much higher than the intensity of the high frequency light pulses, the signal becomes imperceptible and unobservable for the human eye.

With different combination of light sources as illumination unit that can be controlled in intensity and spectrum it is possible to create similar light conditions that a human observer will perceive as the same color. Hence, the optimized illumination spectrum is, in an embodiment, adapted to the normal light conditions to make the illumination during the illumination intervals imperceptible.

To achieve good results it is preferred in an embodiment that said illumination unit is configured to emit light during said illumination intervals that is dominant over the ambient light in a least the wavelength range in which the detection unit receives light.

Preferably, depending on the spectrum of the ambient light or the light sources providing the ambient light the optimal wavelength can be selected. This is preferably achieved by using LEDs with different wavelengths.

In still another embodiment the proposed device further comprises a sensor for sensing ambient light and a control unit for controlling said illumination unit to emit light during said illumination intervals that is dominant over the ambient light in a least the wavelength range in which the detection unit receives light. Thus, the light illumination can be adapted to the ambient lighting conditions and can thus be controlled to be as unobtrusive as possible. The light spectrum is preferably detected automatically with an onboard multi-spectral sensor or an external sensor that can be configured manually. By using an external sensor the illumination spectrum could be identified. This characterization of the spectrum could be used to manually configure the illumination of the device, i.e. not the external sensor but the illumination will preferably be configured in this case.

In still another embodiment said illumination unit is configured to emit light according to a user defined illumination profile in between said illumination intervals. Thus, the provided illumination unit can be used for providing or supporting the “normal” lighting conditions in between the illumination intervals.

In a preferred embodiment said illumination unit is configured to illuminate at least said region of interest during illumination intervals with light, wherein said light is optimized for deriving vital sign information from an input signal by use of remote photoplethysmography from received light reflected from said region of interest, by emitting light having an amplitude such that the variation in the ambient light conditions is insignificant. The minimal required emitted light depends on the frequency and intensity of the disturbing signal. The maximum amount of emitted light before it can be observed by users particularly depends on the flashing frequency and pulse duration intensity and also on the ambient illumination intensity. In an implementation the frequency of the illumination should be such that heart rate signals can be extracted with at least frequencies from 0.25 to 3 Hz (20 to 240 bpm).

Preferably, said detection unit comprises an imaging unit, in particular a camera, such as a video camera, RGB camera and/or infrared camera.

In preferred embodiments the detection unit is configured to generate an input signal for several different wavelength ranges. Thus, depending on the desired vital sign to be derived, the most appropriate one or more input signals may be used for deriving the vital sign information.

Still further, in an embodiment said illumination unit is adapted to set parameters of the light used for illumination the at least one region of interest during said illumination intervals depending on one or more parameters of said at least one region of interest. For instance, depending on the size and/or location of the ROI (e.g. part of the face or the palm of the hand) or depending on the skin color of the living being the brightness and/or frequency of the light can be optimized.

Said ROI(s) may be selected either automatically or manually. For this purpose the proposed device may further comprise a selection unit for automatically selecting said region of interest or allowing a manual selection of said region of interest.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a device for obtaining vital sign information of a living being according to the present invention,

FIG. 2 shows a time diagram illustrating the synchronization of the illumination unit and the detection unit,

FIG. 3 shows a schematic diagram of a second embodiment of a device for obtaining vital sign information of a living being according to the present invention, and

FIG. 4 shows a schematic diagram of a third embodiment of a device for obtaining vital sign information of a living being according to the present invention.

FIG. 5 shows a schematic diagram of a fourth embodiment of a device for obtaining vital sign information of a living being according to the present invention.

### DETAILED DESCRIPTION OF THE INVENTION

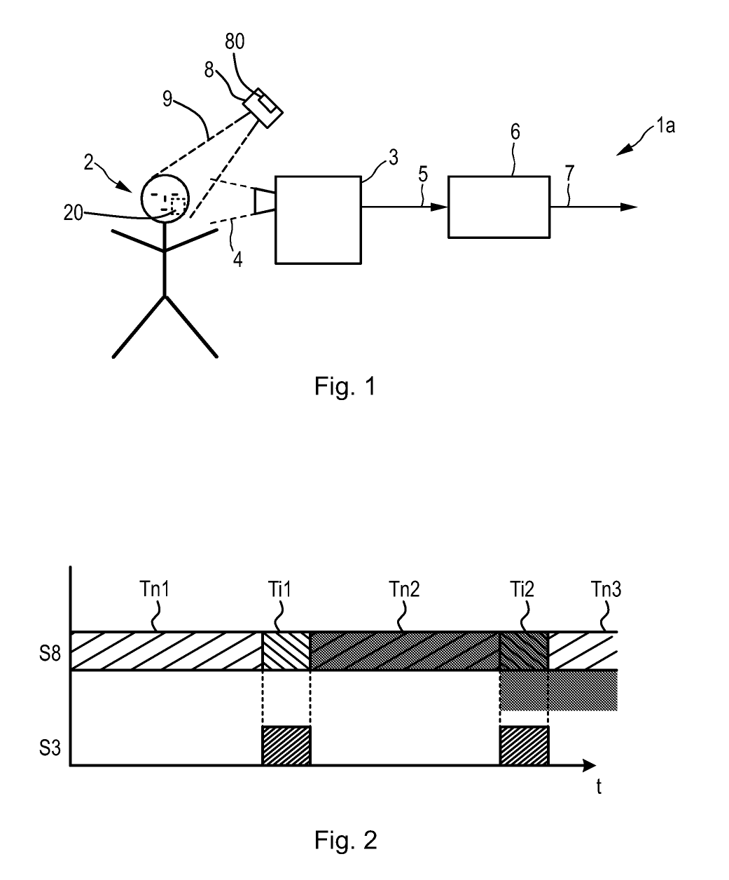

FIG. 1 shows a first embodiment of a device 1a for obtaining vital sign information of a living being 2, e.g. a patient in a hospital, an elderly person monitored in the bed at home or a person doing sports in a fitness club, according to the present invention. The device 1a comprises a detection unit 3 for receiving light 4 in at least one wavelength interval reflected from at least a region of interest of the living being 2 and for generating an input signal 5 from the received light 4. The detection unit 3 is, for instance, configured to register spatio-temporal variations of received light 4, and is preferably an imaging unit for taking images, such as a video camera that substantially continuously or at regular intervals takes images of the living being 2 or at least a region of interest (ROI) 20 of the living being 2.

The device 1a further comprises a processing unit 6 for processing the input signal 5 and deriving vital sign information 7 of said living being 2 from said input signal 5 by use of remote photoplethysmography. The processing unit 6 may e.g. be implemented as software running on a processor or computer, as dedicated hardware or as a mixture of hard- and software. The derivation of vital sign information, e.g. of the heartbeat, respiration signal, SpO2 value, hemoglobin value, etc., is generally known in the art, particularly in the field of remote photoplethysmography, e.g. the above cited paper of Wim Verkruysse et al., which explanation is herein incorporated by reference and shall thus not be explained here in more detail.

The obtained vital sign information 7 is then output from the device 1, e.g. transmitted to a central monitoring station (e.g. a monitoring room of a nurse in a hospital) for display on a monitor, directly displayed next to the living being on a display, or transmitted to a remote control center for further processing and/or display.

The device 1a further comprises an illumination unit 8 for illuminating at least said region of interest 20 during illumination intervals with light 9, wherein said light 9 during said illumination intervals is optimized for deriving vital sign information 7 from an input signal 5 generated by use of remote photoplethysmography from received light 4 reflected from said region of interest 20. Said illumination unit 8 may comprise one or more light sources which are preferably controllable in brightness and/or frequency spectrum of the emitted light. A practical implementation may comprise an one or more arrays of LEDs with specific wavelengths or wavelength ranges. Other embodiments make use of
an LED array with wide spectrum combined with spectral filters with different wavelengths, wherein LEDs are switched with the specific filters;
an LED array with wide spectrum combined with a spectral filter that can adapt its wavelength (electronically/mechanically);
an LED array with broad spectrum and a rotating disk containing filters with different wavelengths (color wheel) like applied in projectors, wherein the wheel position determines the wavelength used;
multiple lasers with specific wavelengths;
an LED array with wide spectrum combined with a spectral filter that can adapt its wavelength (electronically/mechanically);
an LCD screen or other display where the output signal can be controlled, wherein by adding or replacing frames in the video signal and synchronizing the detection unit (camera) the light conditions can be adapted/controlled.

It shall be noted that more than one illumination unit 8 may also be provided, and that other light sources may be present that provide ambient light or lighting conditions desired by a user, e.g. the room light in a hospital room or changing light in a fitness club.

In a passive mode preferably implemented by this embodiment the illumination unit 8 is controlling the period of optimal illumination for measurement of input signals that are optimal for deriving desired vital sign information there from, e.g. for heartbeat measurement. During most of the time the illumination unit 8 shows a user-defined behavior (e.g. is time-varying, low/high intensity, and color) or is even switched off, but for a short periodic illumination period it is configured to provide optimal illumination of at least the ROI 20 for vital sign measurement. The detection unit 3 is able to detect from the obtained light over time, in particular from detected images over time, the periodicity of the light pulses emitted by the illumination unit 8. Thus, the period of optimal illumination can be detected. From the moment when the illumination unit 8 is switched into the “optimal” illumination mode that is optimal for the vital sign measurement (i.e. emits the “optimal” light in the illumination interval) the detection unit 3 starts its measurement until the illumination unit 8 is subsequently switched into its “normal” operation mode, e.g. as defined by the user, or is switched off. When the illumination unit 8 is subsequently again switching into the optimal illumination mode a new measurement (e.g. image acquisition) is started and this sequence is repeated several times or even continuously as long as vital signs shall be obtained.

In an embodiment the detection unit 3 acquires images containing the illumination unit 8. From analysis of the images over time the periodicity of the illumination intervals can be detected to subsequently acquire images (or at least receive light reflected and/or emitted from the ROI 20) only during the illumination intervals to save power and storage space in between said illumination intervals.

FIG. 2 shows a time diagram illustrating the setting S8 of the illumination unit 8 and the setting S3 of the detection unit 3 over time. The illumination unit 8 is alternately switched into the “normal” operation mode during most of the time (normal operation times Tn1, Tn2, Tn3), during which the illumination unit 8 is switched off or contributes to the desired lighting conditions, and into the “optimal” illumination mode during the illumination periods Ti1, Ti2. As can be seen only during the illumination periods Ti1, Ti2 the detection unit 3 is active to receive light from the ROI 20.

In a variation of this embodiment the detection unit 3 continuously detects light from the ROI 20, but the processing unit is configured to only process input signals generated from light received by the detection unit 3 during the illumination periods Ti1, Ti2, but ignores all other input signals. Preferably, input signals are only generated by the detection unit 3 from light received during the illumination periods Ti1, Ti2.

The illumination unit 6 is preferably pre-programmed, e.g. by the user, for which purpose an (optional) interface 80 is provided for programming the illumination unit 8.

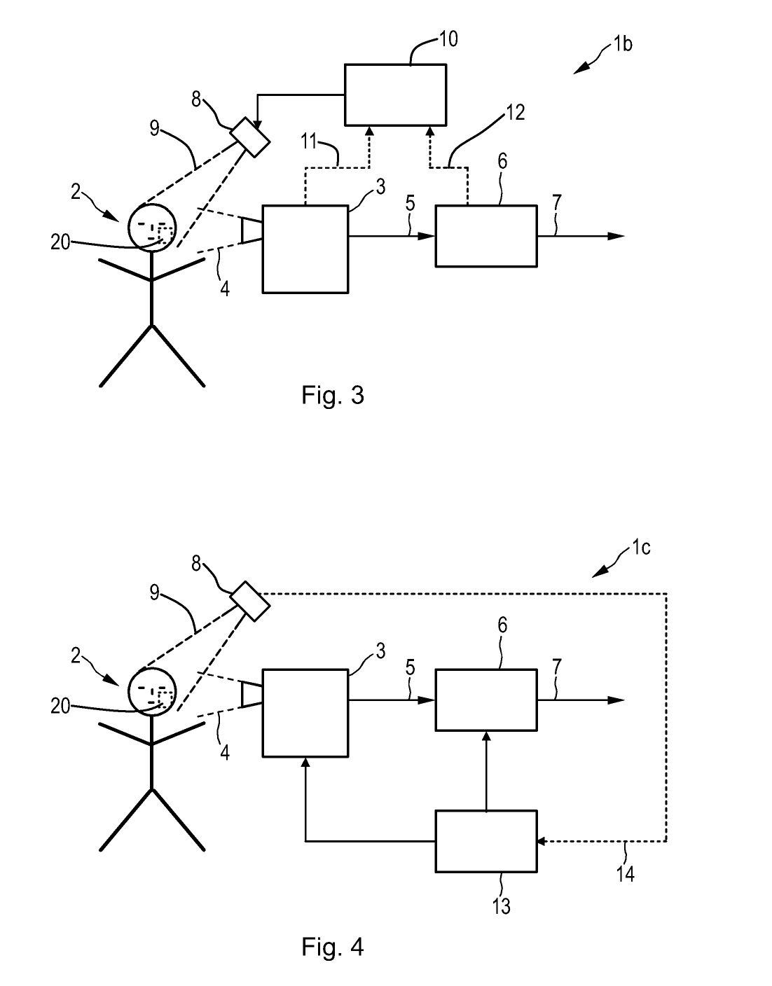

FIG. 3 shows a second embodiment of a device 1b for obtaining vital sign information of a living being 2. In this embodiment employing an active mode a control unit 10 is provided for controlling the illumination unit 6 to illuminate at least said region of interest 20 during said illumination intervals with light optimized for vital sign measurements.

In an implementation the control unit 10 is controlled by a user or a remote operator or is preprogrammed.

Alternatively, the control unit 10 is coupled to the detection unit 3 and/or the processing unit, as indicated in FIG. 3 by broken lines 11 and 12, to synchronize the illumination of said at least one region of interest 20 by said illumination unit 8 with the reception of light and/or generation of input signals by said detection unit 3 and/or with said processing of input signal by said processing unit 6. This further provides the ability to adaptively control the illumination during the illumination intervals based on the obtained vital signs. For instance, if it is recognized by the processing unit 6 that the quality of the derived vital signs is not optimal, the settings of the illumination unit 8 can be modified accordingly to improve the quality by a more optimized illumination of the ROI 20.

FIG. 4 shows a third embodiment of a device 1c for obtaining vital sign information of a living being 2. In this embodiment a control unit 13 is provided for controlling said detection unit 3 to receive light and/or generate input signals only during said illumination intervals and/or for controlling said processing unit 6 to process only portions of input signals 5 generated from light received during said illumination intervals. Preferably, said control unit 13 is coupled to the illumination unit 8, as indicated by broken line 14 to control the detection unit 3 and/or the processing unit 6 based on the illumination intervals, which may thus be variable in time and duration. Alternatively, the control unit 13 may be preprogrammed according to a fixed timing of illumination intervals.

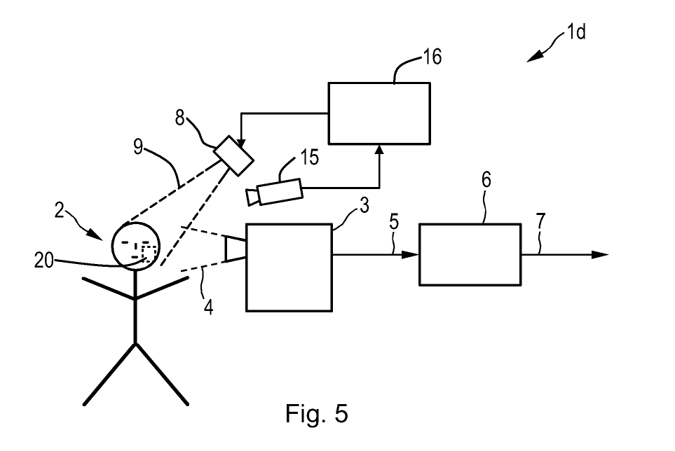

FIG. 5 shows a fourth embodiment of a device 1d for obtaining vital sign information of a living being 2. In this embodiment, a sensor 15 is provided for sensing ambient light, in particular around the living being 2 and particularly in the area of the region of interest 20. Further, a control unit 16 is provided for controlling said illumination unit 8 to emit light during said illumination intervals that is dominant over the ambient light in a least the wavelength range in which the detection unit 3 receives light 4. Thus, based on the detected ambient light the illumination during the illumination intervals can be adapted in real time.

Generally, the illumination shall be performed such that the light is optimized for deriving vital sign information from an input signal by use of remote photoplethysmography from received light reflected from said region of interest wherein light having an amplitude such that the variation in the ambient light conditions is insignificant. The minimal required emitted light generally depends on the frequency and intensity of the disturbing signal. The maximum amount of emitted light before it can be observed by users particularly depends on the flashing frequency and pulse duration intensity and also on the ambient illumination intensity. In an implementation the frequency of the illumination should be such that heart rate signals can be extracted with at least frequencies from 0.25 to 3 Hz (20 to 240 bpm). The sampling of the heart rate signal could be uniform as well as non-uniform.

In a practical embodiment more than 15 frames per second are used to measure the heart rate signal in a fitness application. For a measurement in rest the heart rate is lower and the frame rate and the illumination periodicity can generally be decreased.

It shall be noted that the processing unit 6 and the control units 10, 13, 16 are, in an embodiment, implemented on (the same or separate) processor(s) or computer(s), e.g. on a microprocessor, e.g. by way of a computer program which, when executed, carries out the steps of the proposed processing method.

The present invention may be applied in various applications. Heart rate, breathing rate, and SpO2 are very relevant factors in patient monitoring and home-healthcare where remote heart rate monitoring becomes more and more relevant. Further, the present invention may be applied to register heartbeat in fitness devices. The proposed invention can particularly be applied in any application where camera-based vital signs monitoring is performed with controllable illumination that is changing or with variable light conditions. Normally, the vital signs extraction is extremely challenging and even impossible in some cases, but can now be accurately and reliably achieve.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A device for obtaining vital sign information of a living being, comprising:
a detection unit for receiving light in at least one wavelength interval reflected from at least a region of interest of a living being and for generating an input signal from the received light,
a processing unit for processing the input signal and deriving the vital sign information of said living being from said input signal by use of remote photoplethysmography, and
an illumination unit for illuminating at least said region of interest during illumination intervals with light, wherein said light during said illumination intervals is optimized for deriving vital sign information from an input signal generated by use of remote photoplethysmography from received light reflected from said region of interest.

2. The device as claimed in claim 1, further comprising a control unit for controlling said detection unit to receive light and/or generate input signals only during said illumination intervals.

3. The device as claimed in claim 1, further comprising a control unit for controlling said processing unit to process only portions of input signals generated from light received during said illumination intervals.

4. The device as claimed in claim 1, further comprising a control unit for controlling said illumination unit to illuminate at least said region of interest only during said illumination intervals with light.

5. The device as claimed in claim 1, further comprising a control unit for synchronizing the illumination of said at least one region of interest by said illumination unit with the reception of light and/or generation of input signals by said detection unit and/or with said processing of input signal by said processing unit.

6. The device as claimed in claim 1, wherein said illumination unit is configured to illuminate at least said region of interest during periodic illumination intervals with light, and wherein said detection unit is configured to detect said periodic illumination intervals from received light and to subsequently receive light and/or generate input signals only during said periodic illumination intervals.

7. The device as claimed in claim 1, wherein said illumination unit is configured to control the wavelength of light emitted during said illumination intervals and/or to control the duration of said illumination intervals such that the emitted light during said illumination intervals is invisible or unobtrusive for the human eye.

8. The device as claimed in claim 7, wherein said illumination unit is configured to emit infrared light during said illumination intervals.

9. The device as claimed in claim 7, wherein said illumination unit is configured to emit high frequency light pulses of light in the visible spectral range during said illumination intervals with a low duty cycle.

10. The device as claimed in claim 1, wherein said illumination unit is configured to emit light during said illumination intervals that is dominant over the ambient light in a least the wavelength range in which the detection unit receives light.

11. The device as claimed in claim 1, further comprising a sensor for sensing ambient light and a control unit for controlling said illumination unit to emit light during said illumination intervals that is dominant over the ambient light in a least the wavelength range in which the detection unit receives light.

12. The device as claimed in claim 1, wherein said illumination unit is configured to emit light according to a user defined illumination profile in between said illumination intervals.

13. The device as claimed in claim 1, wherein said illumination unit is configured to illuminate at least said region of interest during illumination intervals with light, wherein said light is optimized for deriving vital sign information from an input signal by use of remote photoplethysmography from received light reflected from said region of interest, by emitting light having an amplitude such that the variation in the ambient light conditions is insignificant.

14. The device as claimed in claim 1, wherein said detection unit comprises an imaging unit.

15. A method for obtaining vital sign information of a living being, comprising:
receiving light in at least one wavelength interval reflected from at least a region of interest of a living being, wherein images of at least said region of interest are acquired from the received light,
generating an input signal from the received light, and
processing the input signal and deriving vital sign information of said living being from said input signal by use of remote photoplethysmography, wherein at least said region of interest is illuminated during illumination intervals with light, wherein said light during said illumination intervals is optimized for deriving vital sign information from an input signal generated by use of remote photoplethysmography from received light reflected from said region of interest.
